(19) United States
(12) Patent Application Publication
(10) Pub. No.: US 2011/0212196 A1
(43) Pub. Date: Sep. 1, 2011

(54) THERAPEUTIC OIL COMPOSITION CONTAINING CARVONE

(75) Inventor: G. Russell Danner, Waterville, ME (US)

(73) Assignee: MAINE CONSERVATION MEDICINE CENTER, Waterville, ME (US)

(21) Appl. No.: 12/714,745

(22) Filed: Mar. 1, 2010

(51) Int. Cl. A61K 31/122; A61K 31/60; A61K 36/45; A61K 36/534

(52) U.S. Cl. 424/747; 514/690; 514/164; 424/769

(57) ABSTRACT

A therapeutic composition having at least one botanical active ingredient, carvone, derived from plants such as mint Mentha sp., caraway Carum carvi or dill Anethum graveolens. Carvone is capable of anesthetizing teleost, elasmobranch, and agnathan fishes as well as amphibians by contact with skin and/or gills. Carvone has two natural enantiomers; both have anesthetic properties on these fishes and amphibians. Carvone in combinations with other sodium channel blockers such as methyl salicylate have a synergistic effect and are more efficient at anesthetizing teleosts, elasmobranchs and agnathan fishes than carvone alone. This invention has value because it offers an opportunity to forumulate fish anesthetizing drugs with a compound that is also documented to be safe for human consumption.

### FIELD OF THE INVENTION

The invention relates to compositions used for anesthesia and other treatment for fishes, shellfish, and amphibians.

### DESCRIPTION OF THE RELATED ART

There are several methods for killing fish that are to be used for human food. One method witnessed was the use of an arc-welding machine by fish hatchery workers to electrocute brook trout. The hatchery workers could not euthanize the fish with chemicals because of the 21-day drug withdrawal period. The electrocution danger to the hatchery workers makes this method unacceptable.

Another method that was tried was the use of an isoeugenol (clove oil) anesthetic oil composition. A particular version of the isoeugenol anesthetic has been marketed under the AQUI-S trademark, and approval for its use was sought under the U.S. Food and Drug Administration's Aquaculture Association Drug Approval Program (“FDA”). However, AQUI-S failed mammalian testing and was not approved by the FDA. Furthermore, the FDA outlawed the use of clove oil because of its isoeugenol component.

Yet another method that has been tried is using carbon dioxide to euthanize fish. It appeared this was the only available method without a slaughter withdrawal period. However, using carbon dioxide to euthanize fish is dangerous, inhumane, and unpleasant. Some fish died during anesthesia, while others jumped about vigorously and injured themselves, bleeding profusely. None of the methods that had been tried were satisfactory. Other marine animals, like shellfish and amphibians, sometimes require a similar treatment for anesthetizing or euthanizing them.

What is needed, therefore, is a therapeutic composition for treating fish, shellfish, and amphibians that is safe, humane, and does not require any drug withdrawal period before human consumption.

### SUMMARY

The invention is a therapeutic composition for treating fish, shellfish, and amphibians that does not have the drawbacks of the prior substances or methods. The composition comprises the active ingredient carvone, found in spearmint oil and dillweed oil with an emulsifier in water, and optionally a sodium channel blocker. These and other features, advantages, and benefits of the present invention will become more apparent with reference to the appended description and claims.

### DETAILED DESCRIPTION

As used in this specification and the claims, the term “treatment” includes but is not limited to anesthetizing and euthanizing. Treatment also includes providing antimicrobial, anti-fungal, and anti-parasitic effects.

Spearmint oil when emulsified in water has anesthetic properties on fishes, shellfish, and amphibians. Dillweed oil when emulsified in water also has anesthetic properties on fishes, shellfish, and amphibians. The purified compound in spearmint oil and dillweed oil capable of anesthetizing fish and amphibians is carvone [C₁₀H₁₄O]. Carvone is found naturally in the seeds and fruits of many plants but is most common in spearmint, dill and caraway. Carvone is a common chemical with many synonyms:

(+)-(4S)-carvone, (+)-(S)-Carvone, (+)-carvone, (−)-2-Methyl-5-isopropenyl-2-cyclohexen-1-one, (−)-Carvone, (R)-2-Methyl-5-(1-methylethenyl)-2-cyclohexen-1-one, (R)-Carvone, (S)-(+)-Carvone, (S)-(+)-p-mentha-6,8-dien-2-one, (S)-2-methyl-5-(1-methylethenyl)-2-Cyclohexen-1-one, (S)-carvone, .alpha.-Carvone, .delta.(sup 6,8)-(9)-Terpadienone-2, .delta.-1-Methyl-4-isopropenyl-6-cyclohexen-2-one, 1-Carvone, 2-Cyclohexen-1-one, 2-methyl-5-(1-methylethenyl)-, 2-Cyclohexen-1-one, 2-methyl-5-(1-methylethenyl)-, 2-Cyclohexen-1-one, 2-methyl-5-(1-methylethenyl)-, (5R)—, 2-Cyclohexen-1-one, 2-methyl-5-(1-methylethenyl)-, (R)—, 2-Cyclohexen-1-one, 2-methyl-5-(1-methylethenyl)-, (R)— (9Cl), 2-Cyclohexen-1-one, 2-methyl-5-(1-methylethenyl)-, (S)—, 2-Cyclohexen-1-one, 2-methyl-5-(1-methylethenyl)-, (theta)-, 2-Methyl-5-(1-methylethenyl)-2-cyclohexen-1-one, 2-Methyl-5-(1-methylethenyl)-2-cyclohexene-1-one, 2-Methyl-5-isopropenyl-2-cyclohexenone, 22327-39-5, 4-07-00-00316 (Beilstein Handbook Reference), 6,8(9)-p-Menthadien-2-one, 6,8-p-Menthadien-2-one, 6485-40-1, 99-49-0, AI3-08877, A13-36200, AIDS-032321, AIDS032321, BRN 1364206, c0627, Carvol, CARVONE, Carvone (natural), D-1-methyl-4-isopropenyl-6-cyclohexen-2-one, D-Carvone, D-Cavone, d-p-Mentha-1(6),8-dien-2-one, D-p-mentha-6,8,(9)-dien-2-one, delta(sup 6,8)-(9)-terpadienone-2, delta-1-Methyl-4-isopropenyl-6-cyclohexen-2-one, EINECS 202-759-5, EINECS 229-352-5, FEMA No. 2249, FEMA Number 2249, HSDB 707, Karvon, L(−)-Carvone, I-1-Methyl-4-isopropenyl-6-cyclohexen-2-one, I-6,8(9)-p-Menthadien-2-one, I-Carvone, I-Carvone (natural), I-p-Mentha-1(6),8-dien-2-one, LS-145724, LS-2355, NCI-055867, NCI60_{—}008753, NCIOpen2_{—}001348, NSC 6275, NSC6275, NSC93738, p-Mentha-6,8-dien-2-one, p-Mentha-6,8-dien-2-one, p-Mentha-6,8-dien-2-one, (−)-, p-Mentha-6,8-dien-2-one, (R)-(−)-, p-Mentha-6,8-dien-2-one.

The common terpenoid ketone, carvone, of the present disclosure can be readily prepared as an oil-in-water emulsion. This emulsion has the ability to anesthetize fishes and amphibians by contact with skin and/or gills. This compound has many potential anthropomorphic uses as cited in (de Carvalho and Da Fonseca 2006). This disclosure deals with carvone's application as an anesthetic for fish and amphibians and other coldblooded animals. The synergistic effect of carvone with other sodium channel blocker botanical has been reported by (Tanaka et al. 1999).

Many different oils were tested for their anesthetic properties. Several had anesthetic properties in fishes at varying doses. However, some oils were very toxic or even melted plastic cups that were used in testing. Some produced a marked twitching behavior that was considered inhumane and unsafe.

It was found that a combination of spearmint oil and wintergreen oil was more effective than either alone in producing acceptable anesthesia. An acceptable composition is one that is safe, humane, produces anesthesia in about two minutes, and recovery in a similar amount of time. Furthermore, combinations of anesthetic compounds often work synergistically to produce a superior anesthetic composition than either alone.

Combinations in the range of 200 mg/L spearmint and 50 mg/L wintergreen were found to work well. However, oil in water in these concentrations tended to separate. Therefore, emulsifiers with a very small micelle were tried. The best emulsifiers were vegetable glycerin and polysorbate 80.

### Examples

1. 400-600 mg/L carvone alone or 75-225 mg/L carvone and 11-32 mg/L methyl salicylate oil-in-water emulsion will anesthetize cold, cool, and warm water teleost fishes. For examples brook trout Salvelinus fontinalis, koi carp Cyprinus carpio, and tilapia Tilapia sp. can be anesthetized by immersion. Fish were anesthetized to handleable condition within 2 minutes, anesthetized to surgical plane within 5 minutes and could be recovered within similar times (2-5 minutes).

2. 750 mg/L carvone and 106 mg/L methyl salicylate oil-in-water emulsion will euthanize cold, cool, and warm water teleost fishes. For examples brook trout Salvelinus fontinalis, koi carp Cyprinus carpio, and tilapia Tilapia sp. can be euthanized by immersion.

3. 400-600 mg/L carvone alone or 75-225 mg/L carvone and 11-32 mg/L methyl salicylate oil-in-water emulsion will anesthetize elasmobranch fishes. For examples spiny dogfish Squalus acanthias can be anesthetized by immersion. Times for anesthesia and recovery were similar to teleost fishes.

4. 750 mg/L carvone and 106 mg/L methyl salicylate oil-in-water emulsion will anesthetize elasmobranch fishes. For examples spiny dogfish Squalus acanthias can be euthanized by immersion.

5. 750 mg/L carvone and 106 mg/L methyl salicylate oil-in-water emulsion will anesthetize amphibians. For examples leopard frog Rana pipiens can be anesthetized and euthanized by placing skin in contact with anesthetic solution. Time to handleable anesthesia was 5 minutes. Time to recovery was about 10 minutes.

Water temperature, pH, and oxygen saturation levels as well as the physical condition of the fish will have an effect on the quality and duration of sedation, anesthesia, and euthanasia. This anesthetic causes a decrease in patient respiration rate and heart rate. Compounds containing both enantiomers of carvone cause more profound apnea and heart rate decrease than either alone.

TABLE 1
Spearmint and Wintergreen
Oil Formula

| Ingredient | Percent |
|---|---|
| Spearmint | 22.8% |
| Wintergreen | 3.2% |
| Vegetable glycerin | 25% |
| Polysorbate 80 | 5% |
| Water | 44% |

Formula for making one liter of fish anesthetic using natural oil ingredients.

TABLE 2
R-(−)-Carvone Formula and
Methyl Salicylate Formula

| Ingredient | Percent |
|---|---|
| R-(−)-carvone | 28.4% |
| Methyl Salicylate | 4.0% |
| Glycerol | 25% |
| Polysorbate 80 | 5% |
| Water | 38% |

Formula for making one liter of fish anesthetic using purified chemical ingredients.

The composition comprising spearmint oil, wintergreen oil, vegetable glycerin, polysorbate 80, and water has the benefits of being natural, kosher, and halal. It was found that spearmint oil is made up of 50-60% L-carvone and wintergreen is largely composed of methyl salicylate. L-carvone is also synthesized directly limonene. L-carvone and methyl salicylate are sodium channel blockers. These two compounds work well together as an anesthetic, and they may together modulate sodium channels in cells, thereby providing a synergistic effect.

In addition to spearmint and L-carvone, a suitable composition might also be made from R-carvone, or the immediate precursors of, or metabolites of, L-carvone, R-carvone, and spearmint oil.

In addition to wintergreen and methyl salicylate, a suitable composition might also be made from other sodium channel blocker botanicals like peppermint oil, menthol or the immediate precursors of or metabolites of menthol.

### REFERENCES

de Carvalho, C. C. C. R., and M. M. R. Da Fonseca. 2006. Carvone: Why and how should one bother to produce this terpene. Food Chemistry 95:413-422.
Tanaka, Y., and coauthors. 1999. Enhancing effects of salicylate on tonic and phasic block of Na⁺ channels by class 1 antiarrhythmic agents in the ventricular myocytes and the guinea pig papillary muscle. Biochimica et Biophysica Acta 1418(2):320-34.

Having fully disclosed the invention, it will be apparent to one of ordinary skill in the art that many changes and modification can be made without departing from the spirit or scope thereof.

I claim:

1. A method for therapeutically treating a fish, shellfish, or amphibian comprising the steps of:
providing an aqueous solution comprising carvone; and
immersing a fish, shellfish or amphibian into the solution.

2. The method of claim 1, wherein the aqueous solution further comprises a sodium channel blocker.

3. The method of claim 2, wherein the sodium channel blocker is one selected from the group consisting of methyl salicylate, wintergreen, peppermint oil, menthol, the immediate precursors of menthol and the immediate metabolites of menthol.

4. The method of claim 1 further comprising an emulsifier.

5. The method of claim 4, wherein the emulsifier is one selected from the group consisting of polysorbate 80, glycerin, and alcohol.

6. A composition for therapeutically treating a fish, shellfish or amphibian comprising:
carvone; and
water.

7. The composition of claim 6 further comprising a sodium channel blocker.

8. The composition of claim 7, wherein the sodium channel blocker is one selected from the group consisting of methyl salicylate, wintergreen, peppermint oil, menthol, the immediate precursors of menthol and the immediate metabolites of menthol.

9. The composition of claim 6 further comprising an emulsifier.

10. The composition of claim 9, wherein the emulsifier is polysorbate 80.

11. The composition of claim 9, wherein the emulsifier is glycerin.

12. The composition of claim 9, wherein the emulsifier is alcohol.

13. The composition of claim 6 wherein the concentration of carvone in water is between 400 mg/L and 600 mg/L.

14. The composition of claim 8 wherein the concentration of carvone in water is between 75 mg/L and 225 mg/L and the concentration of methyl salicylate in water is between 11 mg/L and 32 mg/L.

15. The composition of claim 8 wherein the concentration of carvone in water is 750 mg/L and the concentration of methyl salicylate in water is 106 mg/L.

16. A composition for therapeutically treating a fish, shellfish or amphibian comprising:
spearmint; and
water.

17. The composition of claim 16 further comprising wintergreen.

18. The composition of claim 17 further comprising vegetable glycerin.

19. The composition of claim 18 further comprising polysorbate 80.

20. The composition of claim 19 that is 22.8% spearmint, 3.2% wintergreen, 25% vegetable glycerin, 5% polysorbate 80, and 44% water.
